(19) United States
(12) Patent Application Publication
(10) Pub. No.: US 2022/0156632 A1
(43) Pub. Date: May 19, 2022

(54) IDENTIFYING GENETIC SEQUENCE EXPRESSION PROFILES ACCORDING TO CLASSIFICATION FEATURE SETS

(71) Applicant: International Business Machines Corporation, Armonk, NY (US)

(72) Inventors: LAURA-JAYNE GARDINER, Wirral (GB); RITESH VIJAY KRISHNA, Sale (GB); ANNA PAOLA CARRIERI, Manchester (GB); EDWARD OLIVER PYZER-KNAPP, Runcorn (GB)

(21) Appl. No.: 16/952,153

(22) Filed: Nov. 19, 2020

(51) Int. Cl. G06N 20/00; G16B 25/10; G16B 40/00

(52) U.S. Cl. CPC G06N 20/00; G16B 40/00; G16B 25/10

(57) ABSTRACT

Classifying genetic sequences by receiving genetic sequence data according to sequence features associated with gene expression, determining a genetic sequence feature set, determining a first classification for the genetic sequence feature set according to a machine learning model, defining a causal feature set associated with the first classification for the genetic sequence according to the machine learning model, altering the causal feature set for the genetic sequence, yielding an altered causal feature set, determining a second classification for the altered causal feature set according to the machine learning model, wherein the second classification differs from the first classification, and defining a set of target features, wherein the target features include causal features the altered causal feature set.

### BACKGROUND

The disclosure relates generally to the detection and identification of genetic sequence expression profiles. The disclosure relates particularly to identifying genetic sequence features associated with genetic expression.

Understanding gene expression (also known as the transcriptome) is essential for understanding organism biological development and diseases. Machine learning (ML) has been used for the prediction of transcriptomic profiles using DNA base sequence and/or epigenetic data. DNA base sequence data typically encompasses transcription factor binding sites (TFBS) and/or enhancers. These attributes are thought to contribute to the control of gene expression and attributes such as DNA base sequence features can be identified from pre-existing resources that are widely and publicly available for many species. Current approaches utilize experimental genetic expression data and/or prior knowledge of genetic expression regulatory elements.

### SUMMARY

The following presents a summary to provide a basic understanding of one or more embodiments of the disclosure. This summary is not intended to identify key or critical elements or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatuses and/or computer program products enable the classification of genetic sequence data relating to complex patterns of gene expression.

Aspects of the invention disclose methods, systems and computer readable media associated with classifying genetic sequences according to sequence features associated with gene expression by receiving genetic sequence data, determining a genetic sequence feature set, determining a first classification for the genetic sequence feature set according to a machine learning model, defining a causal feature set associated with the first classification for the genetic sequence according to the machine learning model, altering the causal feature set for the genetic sequence, yielding an altered causal feature set, determining a second classification for the altered causal feature set according to the machine learning model, wherein the second classification differs from the first classification, and defining a set of target features, wherein the target features include causal features from the altered causal feature set.

### BRIEF DESCRIPTION OF THE DRAWINGS

Through the more detailed description of some embodiments of the present disclosure in the accompanying drawings, the above and other objects, features and advantages of the present disclosure will become more apparent, wherein the same reference generally refers to the same components in the embodiments of the present disclosure.

FIG. 1 provides a schematic illustration of a computing environment, according to an embodiment of the invention.

FIG. 2 provides a flowchart depicting an operational sequence, according to an embodiment of the invention.

FIG. 3 depicts a cloud computing environment, according to an embodiment of the invention.

FIG. 4 depicts abstraction model layers, according to an embodiment of the invention.

### DETAILED DESCRIPTION

Some embodiments will be described in more detail with reference to the accompanying drawings, in which the embodiments of the present disclosure have been illustrated. However, the present disclosure can be implemented in various manners, and thus should not be construed to be limited to the embodiments disclosed herein.

In an embodiment, one or more components of the system can employ hardware and/or software to solve problems that are highly technical in nature (e.g., determining a genetic sequence feature set, determining a first classification for the genetic sequence feature set according to a machine learning model, defining a causal feature set for the genetic sequence according to the machine learning model, altering the causal feature set for the genetic sequence, yielding an altered causal feature set, determining a second classification for the altered causal feature set according to the machine learning model, wherein the second classification differs from the first classification, and defining a set of target features, etc.). These solutions are not abstract and cannot be performed as a set of mental acts by a human due to the processing capabilities needed to facilitate genetic sequence classification, for example. Further, some of the processes performed may be performed by a specialized computer for carrying out defined tasks related to classifying genetic sequences. For example, a specialized computer can be employed to carry out tasks related to the classification of genetic sequences, or the like.

Accurately classifying genetic sequences leads to understanding genetic sequence attributes which relate to patterns of gene expression. Identifying sequences associated with patterns of gene expression over the course of a day—circadian rhythms—enable the control and manipulation of such expression patterns through gene editing using tools such as Clustered Regularly Interspaced Short Palindromic Repeat (CRISPR/Cas9). Applications include gene expression therapies and agricultural improvements. Disclosed embodiments enable the classification of genetic sequences associated with patterns of genetic expression.

In an embodiment, the method utilizes a trained machine learning (ML) model to classify genetic sequences. The method trains the model according to the nature of the desired classifications. As an example, for classification of gene sequences or gene promoter sequences associated as either circadian or non-circadian sequences, the method utilizes labeled data including genetic sequences know to be either circadian or non-circadian in their expression, as training and test data for developing the ML classification model.

The method evaluates time series transcriptome data for a set of genes and the set of associated gene promoters. In an embodiment, the method collects associated promoter sequences for input genes as the set of base pairs immediately upstream from the base pair sequence of the gene. For example, the method collects 1500 base pairs upstream from a gene as the promoter sequence for that gene. The transcriptome includes messenger RNA data associated with the activity of a gene/gene promoter. Time series transcriptome data provides data associated with changes in the messenger RNA for the gene/gene promoter over the observed time period. Changes in the transcriptome over time indicate changes in gene/promoter activity or gene/promoter expression over the observed time period.

In an embodiment, transcriptomic analysis of individual genes/promoters of a set of genes/promoters occurred every two hours over a total observation period of 48 hours. The gene/promoter sequences used included known and publicly available gene/promoter sequences. Circadian genes exhibit regular periodic changes in expression—and accompanying changes in the transcriptomic data, over a 24-hour period. Non-circadian gene expression lacks such regular periodic changes in expression. This analysis yielded a training data set of 50,000 genes/promoters with 25,000 labeled as circadian due to transcriptomic data changes over the observed time period and a further 25,000 genes/promoters labeled as non-circadian based upon, the time-series transcriptomic data. The method labeled genes/promoters of the training set according to the expression data observed in the time-series transcriptomic data. Genes/promoters having time-series data including periodic patterns of expression over twenty-four periods labeled as circadian and genes/promoters lacking such periodic patterns of expression labeled as non-circadian. Similarly, the method may be adapted using time-series transcriptomic data for other complex expression patterns to categorize and label training data sets for those complex expression patterns. Once categorized and labeled the set of training genetic sequences need not be generated again.

After using time-series transcriptomic analysis of available gene sequences to generate the training data set, the method processes each gene of the 50,000 gene training data set. The method generates a set of genetic nucleotide subsequences, or k-mer. In an embodiment, the method utilizes k-mer 6 nucleotides in length. Other k-mer lengths, e.g., 4, 8, 10, 12, or more, may be selected and used. For the k-mer, the method generates the set of all possible combinations for nucleotide options of A, T, G, and C (Adenine, Thymine, Guanine, and Cytosine). A total of 4096 possible combinations exist for the 4 nucleotide bases in sets of 6 for the k-mer.

For each of the possible k-mer combinations, the method analyzes the training set of genes and determines the number of occurrences of the k-mer in each gene of the training data set. In an embodiment, the analysis yields a matrix indicating the number of occurrences of each k-mer in each of the genes. For each gene the matrix entries constitute the features of the gene.

In an embodiment, the method counts the number of feature occurrences across the base pair sequence of the gene and additionally counts the feature occurrences across the base pair sequences of the associated gene promoter. The matrix includes the distribution of feature count values for each of the gene and the gene promoter. For this embodiment, the total number of possible features doubles to 8192, 4096 possible features for the gene and 4096 possible features for the gene promoter.

In an embodiment, the method counts the feature occurrences across the combined sequence of the gene and gene promoter. In this embodiment the matrix includes feature count values for each of the 4096 possible features.

In an embodiment, the method reduces the number of features for each gene from the possible 4096 to a smaller number of features such as 100 features. As an example, the method may use a chi squared test to identify the most significant 100 features from the overall set of features in the matrix.

In an embodiment, the method utilizes a classification algorithm to predict classifications for the labeled data of the training set. Exemplary classification algorithms include Logistic Regression, Random Forest, XGBoost, Decision Tree, K-NN (K-nearest neighbors), Gaussian Process, LightGBM (gradient boosting method), and SVM (support vector machine). The method splits the training data set, using 80% of the data for training and 20% of the data for testing the developed algorithm. In this embodiment, the method utilizes a k-nearest neighbors algorithm and achieves an accuracy of 77% in classifying labelled training data utilizing a k value of 2. The method may utilize other k values depending upon the fit of the training data and the accuracy desired in the predictions. The developed model relies solely upon k mer distributions within the training set sequences, without the use of experimental data associated with the genetic sequences. For the example, the trained model classifies feature sets derived from input data sequences as either circadian or non-circadian. The classification dichotomy results from the nature of the training data set. By analogy, labelled training data associated with other complex gene expression patterns yields a model adapted to classify feature sets from input sequences as conforming or not conforming to the complex gene expression patterns.

In practice, the method receives genetic sequence data, processes the sequence data as described yielding a feature set of the sequence and passes the feature set to the classification model for analysis. The model returns a classification of the feature set and associated genetic sequence.

In an embodiment, a user interface, such as a graphical user interface (GUI), provides a user access to the disclosed methods. The method receives genetic sequence data from the user. The user may download, or otherwise provide, publicly available genomic (and epigenetic if available) resources for their species of interest, or else use private user defined datasets. In an embodiment, the method provides links to publicly available genomic databases using application program interfaces (API) associated with such databases. Provided genetic sequence resources will be in the form of genome sequence with gene annotations and/or DNA methylation and/or histone modifications etc.

The method processes the provided sequence data, analyzing the provided data to count the number of occurrences of each of 4096 possible k mer A-G-T-C, nucleotide combinations for k-mers having 6 bases. In an embodiment, the method utilizes epigenetic data to disregard known heavily methylated transcription factor binding sites (TFBS) from amongst the set of features captured in the feature matrix. Ignoring such sites reduces the number of matrix values and limits the matrix of features to features/attributes associated with sequence differences associated with expression differences. The TFBS serve a utilitarian function for expression rather than serving as a gene attribute. The method captures the respective feature counts as a matrix of values associated with each gene analyzed.

The method provides the matrix of features to the trained ML model for classification. The method may reduce the number of matrix values from the full 4096 to a lesser number such as 100 prior to passing the feature set to the ML model for classification. The ML model, such as k-nearest neighbor model, classifies each input feature set. The method provides an explanation for the classification in the form of feature vectors for the input feature set and the nearest neighbors leading to the classification. The method compares the input feature vector and nearest neighbor feature vectors, and the comparison leads to identifying members of a candidate causal feature set—those features of the input feature set most likely to be responsible for the classification of the input as the final classification assigned to it.

In an embodiment, the method ranks the features of the candidate causal feature set using data from the comparison of the input feature vector and the k nearest neighbor feature vectors.

In an embodiment, the method selectively evolves the input gene “in-silico”. For each feature of the candidate causal feature set, the method selectively edits the input genetic sequence, removing the candidate feature from the sequence and from the feature set of the sequence. The method then classifies the edited feature set. The method categorizes edited features which result in a change of classification—for example a feature which alters a sequence from circadian to non-circadian—as members of a target feature set. The method compiles a complete set of target features as all candidate causal features which resulted in a classification change after editing. The complete target feature set provides candidates for actual gene editing to alter the pattern of gene expression of the original input gene. Selectively removing a candidate target feature through a means such as CRISPR/Cas9, should change the expression pattern of the gene as indicated by the change of classification of the edited evolved sequence.

In an embodiment, the final set of target features provides a means of identifying genetic homologs to the input genetic sequence from a first species, in a related species. As an example, a user of the method may apply classification results associated with bread wheat, Triticum aestivum, to a related wheat species such as Triticum durum, or to related grain species such as barley or oat species. As another example, a user may apply gene expression classification results associated with the genome of a first subject to the genome of other subjects of the same species. Application of disclosed embodiments to human genetic sequences presumes that the human donors have consented to, or otherwise opted-in to the use of their genetic sequence data by users of the disclosed methods and systems.

In an embodiment, the method maintains candidate causal feature sets for each classification of the model. In this embodiment, the method selects features from the candidate causal feature set for a first classification for addition through in-silico evolution to input genetic sequences identified as a different classification by the model. Similarly, the method selects features from the candidate causal feature set for a classification for removal through in-silico evolution, from input genetic sequences identified with that classification by the model.

In an embodiment, the method begins the in-silico evolution of the input sequence using the candidate causal feature ranked highest and proceeds from this highest ranked candidate to the lowest ranked candidate. In this embodiment, the method ceases in-silico evolution of candidate causal features after a threshold number of successively ranked candidate causal features fail to result in a classification change; e.g., after 10 successively ranked candidates each fail to result in a classification change, the method ceases the in-silico evolution of the input genetic sequence using the candidate causal features.

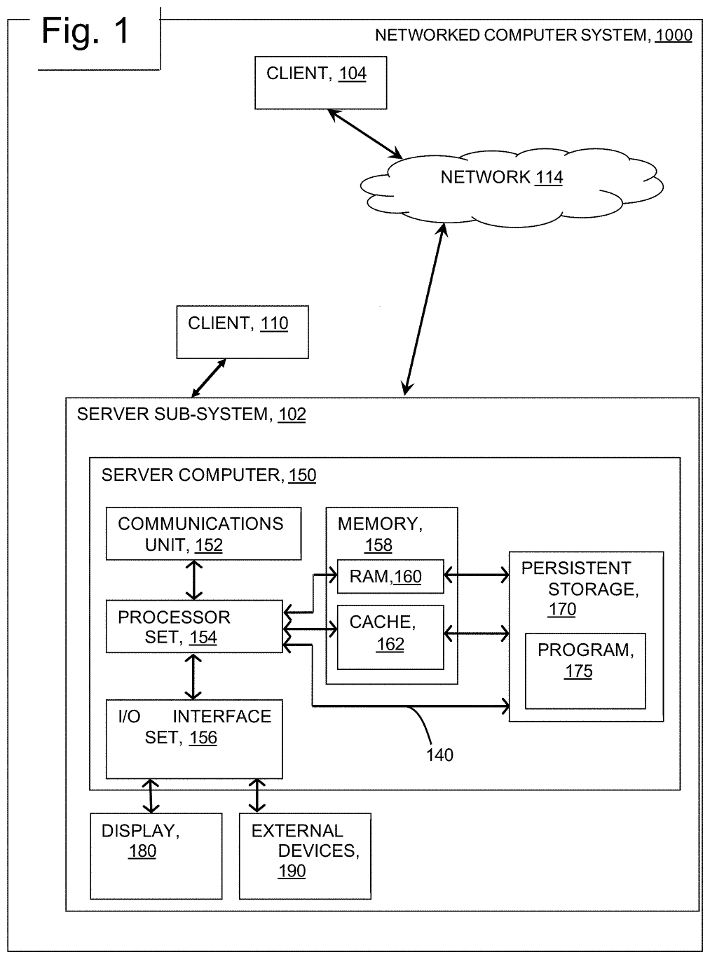

FIG. 1 provides a schematic illustration of exemplary network resources associated with practicing the disclosed inventions. The inventions may be practiced in the processors of any of the disclosed elements which process an instruction stream. As shown in the figure, a networked Client device 110 connects wirelessly to server sub-system 102. Client device 104 connects wirelessly to server sub-system 102 via network 114. Client devices 104 and 110 comprise genetic sequence classification program (not shown) together with sufficient computing resource (processor, memory, network communications hardware) to execute the program. Client devices 104 and 110 serve as user interface devices enabling a user to provide input genetic sequence and epigenetic data to the disclosed methods and system. The client devices 104 and 110 further serve as output devices for the disclosed embodiment to provide output data to the user.

As shown in FIG. 1, server sub-system 102 comprises a server computer 150. FIG. 1 depicts a block diagram of components of server computer 150 within a networked computer system 1000, in accordance with an embodiment of the present invention. It should be appreciated that FIG. 1 provides only an illustration of one implementation and does not imply any limitations with regard to the environments in which different embodiments can be implemented. Many modifications to the depicted environment can be made.

Server computer 150 can include processor(s) 154, memory 158, persistent storage 170, communications unit 152, input/output (I/O) interface(s) 156 and communications fabric 140. Communications fabric 140 provides communications between cache 162, memory 158, persistent storage 170, communications unit 152, and input/output (I/O) interface(s) 156. Communications fabric 140 can be implemented with any architecture designed for passing data and/or control information between processors (such as microprocessors, communications and network processors, etc.), system memory, peripheral devices, and any other hardware components within a system. For example, communications fabric 140 can be implemented with one or more buses.

Memory 158 and persistent storage 170 are computer readable storage media. In this embodiment, memory 158 includes random access memory (RAM) 160. In general, memory 158 can include any suitable volatile or non-volatile computer readable storage media. Cache 162 is a fast memory that enhances the performance of processor(s) 154 by holding recently accessed data, and data near recently accessed data, from memory 158.

Program instructions and data used to practice embodiments of the present invention, e.g., the genetic sequence classification program 175, are stored in persistent storage 170 for execution and/or access by one or more of the respective processor(s) 154 of server computer 150 via cache 162. In this embodiment, persistent storage 170 includes a magnetic hard disk drive. Alternatively, or in addition to a magnetic hard disk drive, persistent storage 170 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 170 may also be removable. For example, a removable hard drive may be used for persistent storage 170. Other examples include optical and magnetic disks, thumb drives, and smart cards that are inserted into a drive for transfer onto another computer readable storage medium that is also part of persistent storage 170.

Communications unit 152, in these examples, provides for communications with other data processing systems or devices, including resources of client computing devices 104, and 110. In these examples, communications unit 152 includes one or more network interface cards. Communications unit 152 may provide communications through the use of either or both physical and wireless communications links. Software distribution programs, and other programs and data used for implementation of the present invention, may be downloaded to persistent storage 170 of server computer 150 through communications unit 152.

I/O interface(s) 156 allows for input and output of data with other devices that may be connected to server computer 150. For example, I/O interface(s) 156 may provide a connection to external device(s) 190 such as a keyboard, a keypad, a touch screen, a microphone, a digital camera, and/or some other suitable input device. External device(s) 190 can also include portable computer readable storage media such as, for example, thumb drives, portable optical or magnetic disks, and memory cards. Software and data used to practice embodiments of the present invention, e.g., genetic sequence classification program 175 on server computer 150, can be stored on such portable computer readable storage media and can be loaded onto persistent storage 170 via I/O interface(s) 156. I/O interface(s) 156 also connect to a display 180.

Display 180 provides a mechanism to display data to a user and may be, for example, a computer monitor. Display 180 can also function as a touch screen, such as a display of a tablet computer.

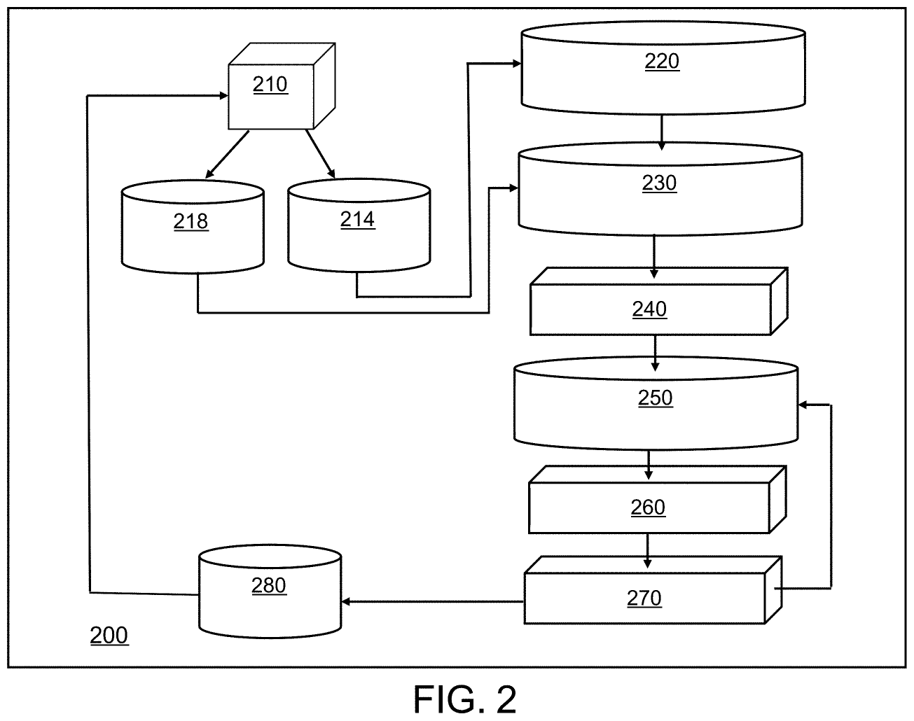

FIG. 2 provides a flowchart 200, illustrating exemplary activities associated with the practice of the disclosure. After program start, a user provides the genetic sequence classification program 175, with genetic sequence data acquired from public sources, private sources, or a combination of public and private sources. The input data includes genome sequence data 214 as well as gene annotations and DNA methylation and/or histone modification data. The input data may further include epigenetic data such as prior domain knowledge of the genome sequence e.g., heavily methylated TFBS sites of the sequence, 218.

At 220, the method of genetic sequence classification program 175 processes input genetic data 214, yielding a matrix of sequence features for the input data. The sequence features include data relating the distribution of possible 6 base k mers within the genome sequence of the input data 214.

At 230 the method of genetic sequence classification program 175 optionally utilizes epigenetic data 218 to reduce the number of entries in the feature matrix from 220. The method removes features associated with known heavily methylated TFBS sites from the matrix or reduces the related matrix entry values to zero.

At 240, the method of genetic sequence classification program 175 classifies or predicts a classification for the input genetic sequence feature set from either 220 or the feature set modified with epigenetic information from 230. The method utilizes a machine learning model trained to classify genetic sequences using a training data set of labeled genetic sequence data related to the desired classifications. As an example, a machine learning model trained using labeled gene sequences associated with each of circadian and non-circadian genetic sequences provides a prediction of either circadian or non-circadian for the provided input feature set.

At 250, the method of genetic sequence classification program 175 uses the classification model explanation for the classification to generate a candidate causal feature set. This set includes those sequence features of the input genetic sequence most likely to have resulted in the model's classification of that input sequence. In an embodiment, the method ranks the members of the candidate feature set from most likely to least likely.

At 260, the method of genetic sequence classification program 175 selectively edits the input genetic sequence and associated input sequence feature set from either 220 or 230. For each member of the candidate causal feature set, the method removes the feature from the input genetic sequence and associated input sequence feature set.

At 270, the method of genetic sequence classification program 175 predicts or classified the edited input feature set using the trained machine learning model. The method passes input features whose removal alters the classification to a target feature set, 280. The method returns to 260 and edits each candidate causal feature in turn, editing the input sequence and associated feature set by only a single candidate causal feature with each iteration.

In an embodiment, the method a general candidate causal feature set for each possible classification of the machine learning model. In this embodiment, at 260, the method either removes a candidate causal feature from the input sequence and input feature from the general candidate causal feature set for the classification of the input sequence, or adds a candidate causal feature from the general candidate causal feature set for a different classification. AS an example, for an input sequence classified as circadian, the method either adds a candidate causal feature from the general candidate causal feature for non-circadian sequences, or removes a candidate casual feature from the candidate causal feature set for the input sequence and input feature set. In this embodiment, the method refines the target feature sets for each possible classification of the machine learning classification model. (Features added from a general causal feature set which result in a change of classification are added to associated target feature set for that classification; e.g., the method adds a feature from the general candidate causal feature set, added to a circadian sequence which results in a re-classification of that sequence to non-circadian, to the target feature set for non-circadian sequences.)

The method provides the sets of target features from 280 to the user via user interface 210. The user may utilize the target features for selectively editing actual genetic sequences for genetic therapies associated with altering gene expression patterns, or to alter plant species genetic expression to enhance agricultural production.

In an embodiment, execution of disclosed methods requires computational resources exceeding those locally available to a user. In this embodiment, the user connects to networked resource including edge cloud and cloud resources to enable a timely execution of the methods.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

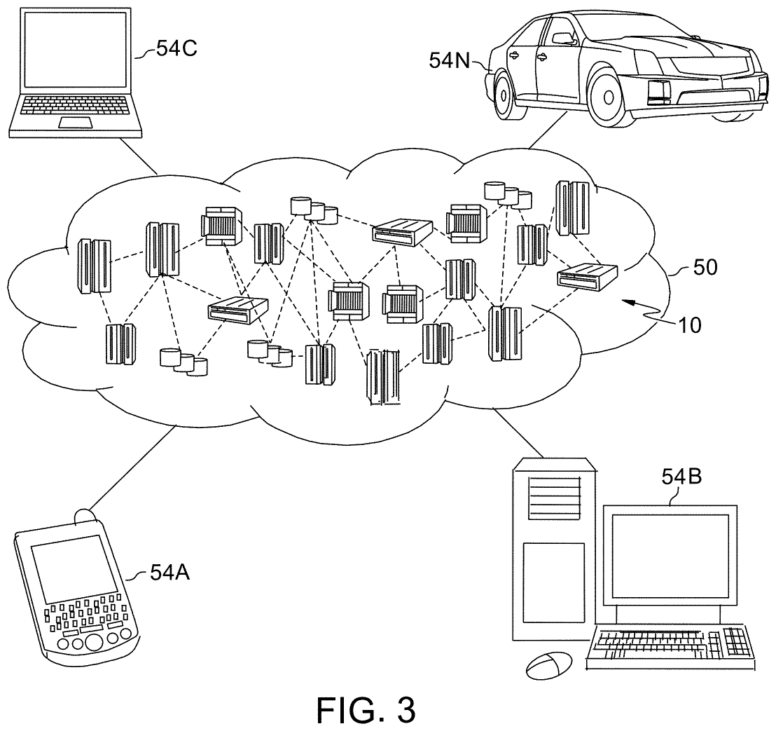

Referring now to FIG. 3, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 3 are intended to be illustrative only and that computing nodes 10 and cloud computing environment 50 can communicate with any type of computerized device over any type of network and/or network addressable connection (e.g., using a web browser).

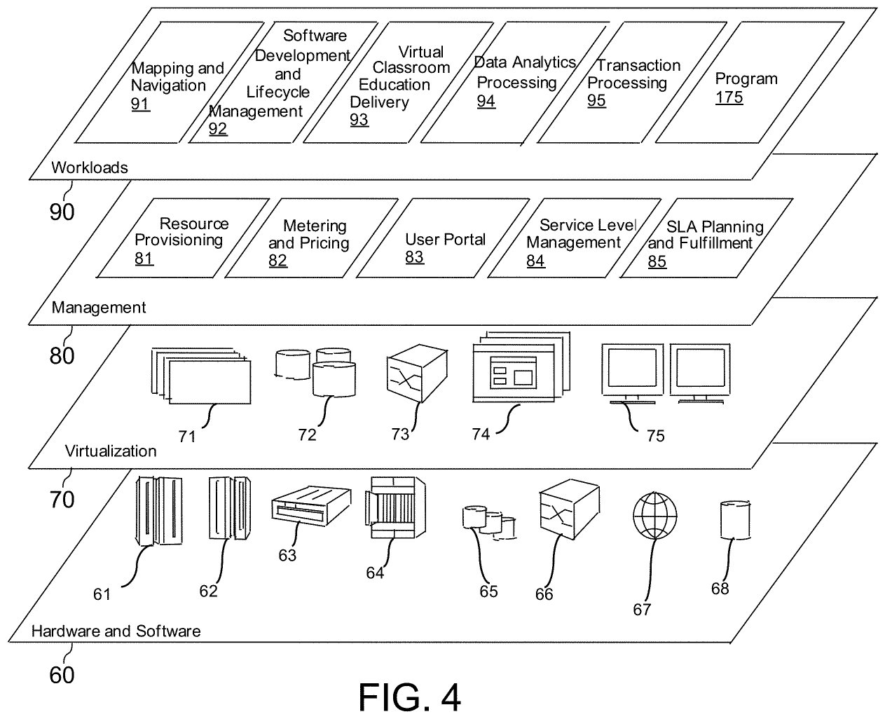

Referring now to FIG. 4, a set of functional abstraction layers provided by cloud computing environment 50 (FIG. 3) is shown. It should be understood in advance that the components, layers, and functions shown in FIG. 4 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and genetic sequence classification program 175.

The present invention may be a system, a method, and/or a computer program product at any possible technical detail level of integration. The invention may be beneficially practiced in any system, single or parallel, which processes an instruction stream. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, or computer readable storage device, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions collectively stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer implemented method for classifying genetic sequences according to sequence features associated with gene expression, the method comprising:
receiving, by one or more computer processors, genetic sequence data;
determining, by the one or more computer processors, a genetic sequence feature set;
determining, by the one or more computer processors, a first classification for the genetic sequence feature set according to a machine learning model;
defining, by the one or more computer processors, a causal feature set associated with the first classification for the genetic sequence according to the machine learning model;
altering, by the one or more computer processors, the causal feature set for the genetic sequence, yielding an altered causal feature set;
determining, by the one or more computer processors, a second classification for the altered causal feature set according to the machine learning model, wherein the second classification differs from the first classification; and
defining, by the one or more computer processors, a set of target features, wherein the target features include causal features from the altered causal feature set.

2. The computer implemented method according to claim 1, wherein determining the genetic sequence feature set comprises determining the genetic sequence feature set according to epigenetic data.

3. The computer implemented method according to claim 1, wherein determining the genetic sequence feature set comprises:
defining a set of possible genetic sequence features; and
determining a distribution of each possible genetic feature within the genetic sequence.

4. The computer implemented method according to claim 1, wherein determining a first classification for the genetic sequence feature set according to a machine learning model comprises determining a circadian/non-circadian classification for the genetic sequence.

5. The computer implemented method according to claim 1, further comprising identifying, by the one or more computer processors, a genetic homolog for the genetic sequence in a related species according to the set of target features.

6. The computer implemented method according to claim 1, further comprising identifying, by the one or more computer processors, editing candidates within the genetic sequence according to the set of target features, the editing candidates associated with altering an expression of the genetic sequence.

7. The computer implemented method according to claim 1, further comprising ranking the set of target features according to a genetic sequence expression prediction.

8. A computer program product for classifying genetic sequences according to genetic sequence features associated with gene expression, the computer program product comprising one or more computer readable storage devices and collectively stored program instructions on the one or more computer readable storage devices, the stored program instructions comprising:
program instructions to receive genetic sequence data;
program instructions to determine a genetic sequence feature set;
program instructions to determine a first classification for the genetic sequence feature set according to a machine learning model;
program instructions to define a causal feature set associated with the first classification for the genetic sequence according to the machine learning model;
program instructions to alter the causal feature set for the genetic sequence, yielding an altered causal feature set;
program instructions to determine a second classification for the altered causal feature set according to the machine learning model, wherein the second classification differs from the first classification; and
program instructions to define a set of target features, wherein the target features include causal features from the altered causal feature set.

9. The computer program product according to claim 8, wherein the program instructions to determine the genetic sequence feature set comprise program instructions to determine the genetic sequence feature set according to epigenetic data.

10. The computer program product according to claim 8, wherein the program instructions to determine the genetic sequence feature set comprise:
program instructions to define a set of possible genetic sequence features; and
program instructions to determine a distribution of each possible genetic feature within the genetic sequence.

11. The computer program product according to claim 8, wherein the program instructions to determine a first classification for the genetic sequence feature set according to a machine learning model comprise program instructions to determine a circadian/non-circadian classification for the genetic sequence.

12. The computer program product according to claim 8, the stored program instructions further comprising program instructions to identify a genetic homolog for the genetic sequence in a related species according to the set of target features.

13. The computer program product according to claim 8, the stored program instructions further comprising program instructions to identify a candidate editing site within the genetic sequence according to the set of target features, the candidate editing site associated with altering an expression of the genetic sequence.

14. The computer program product according to claim 8, the stored program instructions further comprising program instructions to rank the set of target features according to a genetic sequence expression prediction.

15. A computer system for classifying genetic sequences according to genetic sequence features associated with gene expression, the computer system comprising:
one or more computer processors;
one or more computer readable storage devices; and
stored program instructions on the one or more computer readable storage devices for execution by the one or more computer processors, the stored program instructions comprising:
program instructions to receive genetic sequence data;
program instructions to determine a genetic sequence feature set;
program instructions to determine a first classification for the genetic sequence feature set according to a machine learning model;
program instructions to define a causal feature set associated with the first classification for the genetic sequence according to the machine learning model;
program instructions to alter the causal feature set for the genetic sequence, yielding an altered causal feature set;
program instructions to determine a second classification for the altered causal feature set according to the machine learning model, wherein the second classification differs from the first classification; and
program instructions to define a set of target features, wherein the target features include causal features from the altered causal feature set.

16. The computer system according to claim 15, wherein the program instructions to determine the genetic sequence feature set comprise program instructions to determine the genetic sequence feature set according to epigenetic data.

17. The computer system according to claim 15, wherein the program instructions to determine the genetic sequence feature set comprise:
program instructions to define a set of possible genetic sequence features; and
program instructions to determine a distribution of each possible genetic feature within the genetic sequence.

18. The computer system according to claim 15, wherein the program instructions to determine a first classification for the genetic sequence feature set according to a machine learning model comprise program instructions to determine a circadian/non-circadian classification for the genetic sequence.

19. The computer system according to claim 15, the stored program instructions further comprising program instructions to identify a genetic homolog for the genetic sequence in a related species according to the set of target features.

20. The computer system according to claim 15, the stored program instructions further comprising program instructions to identify a candidate editing site within the genetic sequence according to the set of target features, the candidate editing site associated with altering an expression of the genetic sequence.
